(19) United States
(12) Patent Application Publication
(10) Pub. No.: US 2009/0285889 A1
(43) Pub. Date: Nov. 19, 2009

(54) MODIFIED RELEASE FORMULATIONS OF DIHYDROPYRIDINE COMPOUNDS AND METHODS OF MAKING SAME

(75) Inventors: Vittorino Raveli, Milano (IT); Subraman Rao Cherukuri, Vienna, VA (US); Revanth Babu Mutyala, Frederick, MD (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Capricom Pharma Inc., Frederick, MD (US)

(21) Appl. No.: 12/466,105

(22) Filed: May 14, 2009

Related U.S. Application Data
(60) Provisional application No. 61/071,702, filed on May 14, 2008.

(51) Int. Cl. A61K 9/28; A61K 31/4422

(52) U.S. Cl. 424/474; 514/356

(57) ABSTRACT

The invention relates to a modified release (e.g., extended release) tablet composition for oral administration comprising: (a) a dihydropyridine compound or a prodrug or salt thereof; (b) a first release control agent; (c) a second release control agent; and (d) a third release control agent; wherein said second release control agent has a higher viscosity than said first release control agent. The invention also relates to methods for making such modified release (e.g., extended release) tablet compositions. After tableting, such compositions exhibit a release profile that is approximately zero order without the need of a coating.

### CROSS-REFERENCE TO RELATED APPLICATIONS

This application claims the benefit of U.S. Provisional Application Ser. No. 61/071,702, filed May 14, 2008, which is incorporated by reference in its entirety herein.

### FIELD OF THE INVENTION

The subject matter disclosed herein relates generally to modified release (e.g., extended release) formulations of dihydropyridine compounds and methods of making those formulations. In a preferred embodiment, the invention relates to modified release (e.g., extended release) formulations comprising the dihydropyridine compound nifedipine.

### BACKGROUND

Active compounds from the dihydropyridine class of substances and their use as cardiac and circulatory agents are well known, See, e.g., U.S. Pat. Nos. 4,892,741 and 5,264,446. The formulations described in U.S. Pat. No. 4,892,741 are in the form of press-coated tablets (i.e., a tablet within a tablet) comprising a core that rapidly releases nifedipine and a nifedipine-containing coating that is press-coated onto the core. The coating, unlike the core, slowly releases nifedipine. In some embodiments, the tablet is further coated with a “gastric juice-resistant layer.” The formulations described in U.S. Pat. No. 5,264,446 explore the effects of varying the specific surface area of nifedipine crystals on the bio-availability of nifedipine. Both patents purport to address difficulties that frequently appear in devising formulations for oral administration of these potent active compounds such as their very low solubility, light-sensitivity and their bioavailability, which is frequently hampered by their poor solubility and absorbability in biological systems. One major drawback of the technology described and claimed in these two patents, however, includes the variable release of nifedipine from the inner core from tablet to tablet. The variable release can be ascribed to the fact that the tablets described and claimed in those two patents are press-coated tablets. What often occurs with such press-coated tablets is that the core is not always located perfectly at the center of the press-coating. This results in a press-coated tablet having a core that is sometimes slightly off center, which leads to a tablet that exhibits variable release of nifedipine from tablet to tablet, because the coating is effectively thicker in some areas and thinner in others from tablet to tablet. Accordingly, novel formulations of dihydropyridine compounds are needed that address such difficulties. The claimed formulations overcome such difficulties.

### SUMMARY OF THE INVENTION

The formulations of dihydropyridine compounds, and the methods of making such formulations disclosed herein, solve the problems of solubility and bioavailability by providing a dihydropyridine compound formulation that is dry blended prior to tableting with at least three release control agents in the absence of a solvent. At least two of the three release control agents are water soluble polymers that form solutions of different viscosities in water. In a typical embodiment of the present invention, one of the release control agents comprises a hypromellose that forms a low viscosity (e.g., 50-500 cPs) solution in water, another of the release control agents comprises a hypromellose that forms a high viscosity (e.g., 3,000-6,000 cPs) solution in water, and a third release control agent comprises an osmotic agent, an emulsifier, a water-soluble sugar, a pH-dependent releasing agent, or a mixture thereof. After tableting, the resulting formulation exhibits a release profile that is approximately zero order. Any cardiac and circulatory agent can be formulated according to the methods disclosed herein. A preferred cardiac and circulatory agent is the dihydropyridine compound nifedipine. In a first aspect, therefore, the invention relates to an modified release (e.g., extended release) tablet composition for oral administration comprising:

(a) a dihydropyridine compound or a prodrug or salt thereof;
(b) a first release control agent;
(c) a second release control agent; and
(d) a third release control agent;
wherein the second release control agent has a higher viscosity in solution than the first release control agent.

In a second aspect, the invention relates to a process for making the tablet composition according to the first aspect, comprising:

(a) providing a composition comprising the dihydropyridine compound or a prodrug or salt thereof first release control agent, second release control agent, and third release control agent and dry blending the composition;
(b) granulating the dry blend with a polymer, a cellulosic material, polyethylene glycol, or mixtures thereof, to give a granulate; and
(c) tableting the granulate composition to give a tablet.

In another aspect, the invention relates to a tablet composition for oral administration comprising:

(a) a dihydropyridine compound or a prodrug or salt thereof,
(b) a first release control agent;
(c) a second release control agent; and
(d) an osmotic agent;
wherein the dihydropyridine compound or a prodrug or salt thereof is provided in a micronized, crystalline or amorphous form when the tablet is formed; and
wherein, in vitro at a pH of less than 7 and in the presence of 1% sodium lauryl sulfate, the composition releases from about 25% to about 50%, or, e.g., from about 25% to about 55% of the dihydropyridine compound or a prodrug or salt thereof after 6 hours and not less than about 65% or not less than 75% of the dihydropyridine compound or a prodrug or salt thereof after 12 hours.

In another aspect, the invention relates to a tablet composition for oral administration comprising.

(a) a dihydropyridine compound or a prodrug or salt thereof;
(b) a first release control agent;
(c) a second release control agent; and
(d) an osmotic agent;
wherein the dihydropyridine compound or a prodrug or salt thereof is provided in a micronized, crystalline or amorphous form when the tablet is formed; and
wherein, in vitro at a pH of greater than 7, the composition releases from about 10% to about 20% of the dihydropyridine compound or a prodrug or salt thereof after 6 hours and from about 25% to about 35% of the dihydropyridine compound or a prodrug, or salt thereof after 12 hours.

In still another aspect, the invention relates to a tablet composition for oral administration comprising:

(a) a dihydropyridine compound or a prodrug or salt thereof,
(b) a first release control agent;
(c) a second release control agent; and
(d) an osmotic agent;
wherein the dihydropyridine compound or a prodrug or salt thereof is provided in a micronized, crystalline or amorphous form when the tablet is formed; and
wherein, in vitro at a pH of 1.2, in the presence of 1% sodium lauryl sulfate, using a USP Type II apparatus operating at 50 rpm and comprising 900 mL of water, the composition releases from about 1% to about 10% of the dihydropyridine compound or a prodrug or salt thereof after one hour; from about 10% to about 30% after four hours; from about 40% to about 65% after eight hours; and from about 70% to about 90% after 12 hours.

In yet another aspect, the invention relates to a tablet composition for oral administration comprising:

(a) a dihydropyridine compound or a prodrug or salt thereof,
(b) a first release control agent;
(c) a second release control agent; and
(d) an osmotic agent;
wherein the dihydropyridine compound or a prodrug or salt thereof is provided in a micronized, crystalline or amorphous form when the tablet is formed; and
wherein, in vitro at a pH of 4.5, in the presence of 1% sodium lauryl sulfate, using a USP Type II apparatus operating at 50 rpm and comprising 900 mL of water, the composition releases from about 1% to about 15% of the dihydropyridine compound or a prodrug or salt thereof after one hour; from about 7% to about 20% after two hours; from about 20% to about 40% after four hours; from about 40% to about 75% after eight hours; and from about 70% to about 90% after 12 hours.

In still another aspect, the invention relates to a tablet composition for oral administration comprising:

(a) a dihydropyridine compound or a prodrug or salt thereof;
(b) a first release control agent;
(c) a second release control agent; and
(d) an osmotic agent;
wherein the dihydropyridine compound or a prodrug or salt thereof is provided in a micronized, crystalline or amorphous form when the tablet is formed; and
wherein, in vitro at a pH of 6.8, in the presence of 1% sodium lauryl sulfate, using a USP Type II apparatus operating at 50 rpm and comprising 900 mL of aqueous phosphate buffer, the composition releases from about 1% to about 10% of the dihydropyridine compound or a prodrug or salt thereof after one hour; from about 7% to about 20% after two hours; from about 20% to about 40% after four hours; from about 50% to about 80% after eight hours; and from about 75% to about 90% after 12 hours.

### BRIEF DESCRIPTION OF THE DRAWINGS

These and other features and aspects of exemplary embodiments of the invention will become better understood when reading the following detailed description with reference to the accompanying drawings wherein:

FIG. 1 is a plot of cumulative percentage of drug release versus time in hours for nifedipine formulations made according to the present invention and commercially available nifedipine formulations at pH 1.2, 6.8, and 7.5;

FIG. 2 is a plot of cumulative percentage of drug release versus time in hours for nifedipine formulations made according to the present invention at pH 1.2, 4.5, and 6.8; and

FIG. 3 is a plot of cumulative percentage of drug release at pH 4.5 versus time in hours for nifedipine formulations made according to the present invention and commercially available nifedipine formulations (e.g., ADALAT® CC, Bayer Pharmaceuticals Corp., West Haven, Conn.).

### DETAILED DESCRIPTION

### Definitions

In describing and claiming the present invention, the following terminology will be used in accordance with the definitions set forth below.

The singular forms “a,” “an,” and, “the” include plural referents unless the context clearly dictates otherwise. Thus, for example, reference to “a drug” includes reference to one or more of such drugs, and reference to “an excipient” includes reference to one or more of such excipients.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients.

As used herein, “active agent,” “bioactive agent,” “pharmaceutically active agent,” and “pharmaceutical,” may be used interchangeably to refer to an agent or substance that has a measurable, specified, or selected physiologic activity when administered to a subject in a significant or effective amount. It is to be understood that the term “drug” is expressly encompassed by the present definition, since many drugs and prodrugs are known to have specific physiologic activities. These terms of art are well-known in the pharmaceutical, and medicinal arts.

As used herein, “dihydropyridine compound” refers to a compound having the following dihydropyridine core, regardless of substitution about the core:

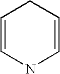

Exemplary dihydropyridine compounds that have the dihydropyridine core include, without limitation, amlodipine, aranidipine, azelnidipine, barnidipine, benidipine, cilnidipine, clevidipine, cronidipine, darodipine, dexniguldipine, efonidipine, elnadipine, elgodipine, felodipine, flordipine, furnidipine, iganidipine, isradipine, lacidipine, lemildipine, lercanidipine, manidipine, mesuldipine, nicardipine, nifedipine, niguldipine, nimodipine, niludipine, nilvadipine, nimodipine, nisoldipine, nitrendipine, olradipine, oxodipine, palonidipine, pranidipine, sagandipine, sornidipine, teludipine, tiamdipine, trombodipine, watanidipine, and mixtures thereof or prodrugs or salts thereof.

As used herein, “hypromellose” refers to hydroxypropyl methylcellulose.

The term “dihydropyridine compound” may also be used from time to time herein to refer to not only dihydropyrdine compounds, but also to encompass related compounds, such as analogs and homologs thereof, salts, such as acid addition salts thereof, prodrugs, enantiomers and metabolites thereof, as well as mixtures thereof, as dictated by the context of its use. When referring to individual specific related compounds, or groups of compounds such as the acid addition salts, the specific technical name of each compound or molecule will be used, or the group will be specifically named, such as “dihydropyridine compound salts.”

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, “blood level” may be used interchangeably with terms such as blood plasma concentration, plasma level, plasma concentration, serum level, serum concentration, serum blood level and serum blood concentration.

As used herein, “zero order release” refers to a rate of drug release that is substantially constant and can be approximated by the formula (I):

R(t)=k₀

where R is the rate of release of the drug(s) as a function of time t and is equal to the zero order rate constant k₀. See, Chemg-ju Kim, Controlled Release Dosage Form Design 278 (Technomic Publishing Co., Inc.) (2000), the entirety of which is incorporated by reference as if fully set forth herein.

As used herein, “approximately zero order release” refers to a rate of drug release that is substantially constant.

As used herein, “oral dosage form” and the like refers to a formulation that is ready for administration to a subject through the oral route of administration. Examples of known oral dosage forms, include without limitation, tablets, minitablets, capsules, caplets, powders, pellets, granules, etc. In some aspects, powders, pellets, granules, tablets, and minitablets may be coated with a suitable polymer or a conventional coating material to achieve, for example, greater stability in the gastrointestinal tract, or to achieve the desired rate of release. Moreover, capsules containing a powder, pellets, minitablets, or granules may be further coated. Tablets and caplets may be scored to facilitate division of dosing. Alternatively, the dosage forms of the present invention may be unit dosage forms wherein the dosage form is intended to deliver one therapeutic dose per administration. Particular embodiments or groups of embodiments may be expressly limited to subsets of these dosage forms.

As used herein, “sachet” refers to a small, sealed packet containing a quantity of material, which is typically a single-use quantity.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, e.g., Meiner and Tonascia, “Clinical Trials: Design, Conduct, and Analysis,” Monographs in Epidemiology and Biostatistics, Vol. 8 (1986), incorporated herein by reference.

As used herein, “pharmaceutically acceptable carrier” “carrier,” and “excipient” may be used interchangeably, and refer to any inert and pharmaceutically acceptable material that has substantially no biological activity, and makes up a substantial part of the formulation.

The term “admixed” means that the drug and/or other ingredients can be dissolved, dispersed, or suspended in the carrier. In some cases, the drug may be uniformly admixed in the carrier.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

The term “modified release” as used herein refers to the drug release that is different from an immediate release. Typically, in an immediate release dosage form, about more than 80% of the drug is released from the dosage form in vitro within about 2 hours. This release may be measured in terms of dissolution of the drug in the dissolution medium. In one aspect, the release is measured under USP conditions, i.e., where the pH is maintained at 1.2 for 2 hours, followed by a pH of 6.8 for the rest of the time. In another aspect, the release is measured at a pH of 1.2 for the entire period of measurement. Other conditions suitable for measurement of modified release are described herein. Examples of such modified release include extended release, sustained release, slowrelease, delayed-release, pulsatile release, etc., which terms are generally known in the art and to the extent they mean a release other than an immediate release. For example, an extended release dosage form is one that allows at least a two-fold reduction in dosing frequency as compared to that drug presented as an immediate release dosage form.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

The Invention

The present invention provides modified release dihydropyridine compound-containing dosage forms with certain desirable in vitro dissolution properties and in vivo characteristics. In one aspect, the invention provides methods for formulating a modified release tablet, minitablet (e.g., a 1 mm×3 mm; a 3 mm×3 mm; or a 1 mm×7 mm minitablet), or capsule comprising a dihydropyridine compound, where the dihydropyridine compound or a prodrug or salt thereof is substantially homogeneously dispersed throughout the tablet. In some embodiments, the dihydropyridine compounds of the present invention can be combined with other drug(s). Non-limiting examples of such drugs include beta blockers (e.g., acebutolol, alprenolol, atenolol, bopindolol, metoprolol, nadolol, oxprenolol, pindololpropanolol, practolol, propanolol, sotalol, and timolol) and anti-arythmics (e.g., quinidine, ajmaline, procainamide, disopyramide, propafenone, tocainide, phenytoin, aprindine, mexiletine, flecainide, lorcainide, propafenone, sotalol, amiodarone, verapamil, and diltiazem).

In a preferred embodiment, the invention provides for an modified release (e.g., extended) release tablet composition for oral administration comprising:

(a) a dihydropyridine compound or a prodrug or salt thereof,
(b) a first release control agent;
(c) a second release control agent; and
(d) a third release control agent;
wherein said second release control agent has a higher viscosity in solution than said first release control agent.

In some embodiments, the dihydropyridine compound is provided in a micronized, crystalline or amorphous form when the tablet is formed. In other embodiments, the dihydropyridine compound is provided in a micronized mixture of the amorphous and the crystalline form when the tablet is formed. Regardless of whether the dihydropyridine compound is in the crystalline or amorphous form (or as a mixture of each form), the micronized dihydropyridine compound comprises particles such that about 95% of the particles are from less than about 25 microns to about 1 micron, or from less than about 15 microns to about 5 microns. In some embodiments, the surface area of the micronized dihydropyridine compound (crystalline, amorphous, or mixtures of each form) is at least about 4 m²/gram, e.g., at least about 5 m²/gram, or at least about 10 m²/gram.

The dihydropyridine compound can comprise from about 10% to about 50% by weight of the total composition, e.g., from about 10% to about 40% by weight of the total composition, from about 20% to about 30% by weight of the total composition, or from about 25% to about 35% by weight of the total composition.

In some embodiments, the first release control agent comprises a cellulosic material. As used herein, “cellulosic material” refers to derivatized celluloses such as, for example, methylcellulose, hydroxypropyl cellulose (HPC), hydroxyethyl cellulose (HEC), hypromellose, or mixtures of such derivatized celluloses. In one embodiment, the cellulosic material in the first release control agent forms a low viscosity solution in water (2% solution in water). The viscosity of such a solution ranges from about 50-500 cPs, e.g., from about 50 to about 120 cPs, from about 80 to about 120 cPs, or from about 100 to about 120 cPs. A non-limiting example of the first release control agent is hypromellose such as METHOCEL™ K100 Premium LV (NW 26,000 g/mol; Dow Chemical). The first release control agent can comprise from about 10% to about 50% by weight of the total composition, e.g., from about 10% to about 40% by weight of the total composition, from about 20% to about 30% by weight of the total composition, or from about 25% to about 35% by weight of the total composition.

In some embodiments, the second release control agent comprises a cellulosic material that may be the same or different than the cellulosic materials comprised in the first release control agent. In one embodiment, the cellulosic material in the second release control agent forms a high viscosity solution in water (2% solution in water). The viscosity of such a solution ranges from about 2,500 to about 100,000 cPs, e.g., from about 10,000 to about 100,000 cPs, from about 3,000 to about 10,000 cPs, or from about 3,000 to about 6,000 cPs. A non-limiting example of the second release control agent is hypromellose such as METHOCEL™ K4M Premium (MW 86,000 g/mol; Dow Chemical). The second release control agent can comprise from about 1% to about 30% by weight of the total composition, e.g., from about 1% to about 20% by weight of the total composition, from about 5% to about 15% by weight of the total composition, or from about 5% to about 10% by weight of the total composition.

In some embodiments, the molar ratio of the first release control agent to the second release control agent is about 100:1, about 50:1; about 30:1, about 20:1, or about 10:1. In one preferred embodiment, the ratio of the first release control agent to the second release control agent is about 11:1. In other embodiments, the molar ratio of the first release control agent to the second release control agent to the third release control agent is about 200:20:1, or about 180:10:1, or 160:10:1.

Examples of combinations of first release control agents and second release control agents contemplated for use in the present invention include those listed in Table 1, below.

TABLE 1

| | Molecular Weight | | Molecular Weight |
|---|---|---|---|
| | (g/mol)/Apparent | | (g/mol)/Apparent |
| Second Release Control | Viscosity | First Release Control | Viscosity |
| Agent | (cP)/Substitution Type | Agent | (cP)/Substitution Type) |
| Methocel (HPMC) K4M | 89,000 (n)/4,000¹/2208⁵ | Methocel (HPMC) K3 | 9,000 (n)/3/2208⁵ |
| Premium CR | | Premium LV | |
| Methocel (HPMC) K15M | 200,000 (n)/15,000¹/2208⁵ | Methocel (HPMC) K100 | ~50,000 (n)/100/2208⁵ |
| Premium CR | | Premium LV | |
| Methocel (HPMC) | 400,000 (n)/100,000¹/2208⁵ | Natrosol (HEC) G Pharm | 300,000 (w)/325/NA |
| K100M Premium CR | | | |
| Klucel (HPC) HXF | 1,150,000 (w)/2,250²/NA | Natrosol (HEC) L Pharm | 90,000/11/NA |
| Methocel (HPMC) E4M | 93,000 (n)/4,000¹/2910⁶ | Klucel (HPC) GF Pharm | 370,000 (w)/275/NA |
| Premium CR | | | |
| Klucel (HPC) HF Pharm | 850,000 (w)/5,250²/NA | Klucel (HPC) JF Pharm | 140,000 (w)/275³/NA |
| Klucel (HPC) MF Pharm | 720,000 (w)/5,500¹/NA | Klucel (HPC) LF Pharm | 95,000 (w)/110³/NA |
| Natrosol (HEC) M Pharm | 1,000,000 (w)/30,000¹/NA | Klucel (HPC) EF Pharm | 80,000 (w)/450⁴/NA |
| Natrosol (HEC) HX | 1,300,000 (w)/100,000¹/NA | Methocel (HPMC) F50 | 45,000 (n)/50/2906⁶ |
| Pharm | | Premium | |
| Natrosol (HEC) HHW | 1,300,000 (w)/100,000¹/NA | Methocel (HPMC) E50 | 45,000 (n)/50/2910⁷ |
| Pharm | | Premium LV | |
| Natrosol (HEC) HHX | 1,300,000 (w)/100,000¹/NA | Methocel (HPMC) E15 | 30,000 (n)/15/2910⁷ |
| Pharm | | Premium LV | |
| Methocel (HPMC) E10M | 100,000 (w)/10,000¹/2910 | Methocel (HPMC) E6 | 20,000 (n)/6/2910⁷ |
| Premium CR | | Premium LV | |
| | | Methocel (HPMC) E5 | 10,000 (n)/5/2910⁷ |
| | | Premium LV | |
| | | Methocel (HPMC) E3 | 9,000 (n)/3/2910⁷ |
| | | Premium LV | |
| | | Methocel (Methyl | 13,500 (n)/15/NA |
| | | cellulose) A15 Premium | |
| | | LV | |
| | | Methocel (Methyl | 41,000 (n)/400/NA |
| | | cellulose) A4C Premium | |
| | | Methocel (Methyl | 63,000 (n)/1500/NA |
| | | cellulose) A15C Premium | |

(n) is number average molecular weight.
(w) is weight average molecular weigh.
¹2% percentage of solids in the aqueous solution.
²1% percentage of solids in the aqueous solution.
³5% percentage of solids in the aqueous solution.
⁴10% percentage of solids in the aqueous solution.
⁵Methoxy percent: 19.0 (min)/24.0 (max); hydroxypropoxy percent: 4.0 (min)/12.0 (max).
⁶Methoxy percent: 27.0 (min)/30.0 (max); hydroxypropoxy percent: 4.0 (min)/7.5 (max) . . .
⁷Methoxy percent: 28.0 (min)/30.0 (max); hydroxypropoxy percent: 7.0 (min)/12.0 (max) . . .

One preferred combination of first release control agent and second release control agent is HPMC K100LV and HPMC K4M, respectively. Another preferred combination includes HPMC substitution type 2208 with a nominal viscosity of 100 cP and HPMC substitution type with a nominal viscosity of 4000 cP. The skilled artisan can develop additional combinations of first release control agent and second release control agent by choosing from the first release control agents and second release control agents listed in Table 1.

In some embodiments, the third release control agent comprises an osmotic agent, an emulsifier, e.g., an emulsifier that has a high hydrophilic lipophilic balance (HLB) (e.g., an HLB that is greater than 2, greater than 4, greater than 5, or greater than 10), a water-soluble sugar, or a pH-dependent releasing agent, or mixtures thereof. While not being bound by any particular theory, it is believed that the third release control agent acts as an expedient for the release of the nifedipine. As used herein, “expedient” refers to an agent that expedites the release (i.e., increases the rate of release) of nifedipine, in vitro and/or in vivo, relative to the release of nifedipine from a formulation that lacks the expedient.

Exemplary osmotic agents include, without limitation, sodium chloride, potassium monophosphate, fumaric acid, and mixtures thereof. Exemplary emulsifiers include, without limitation, block copolymers based on ethylene oxide and propylene oxide (e.g., Pluronics®), polyoxylated sorbitan-based compounds (e.g., polysorbate 20, polysorbate 21, polysorbate 40, polysorbate 60, polysorbate 61, polysorbate 65, polysorbate 80, and polysorbate 81), fatty acid salts (e.g., sodium stearate, zinc stearate, and other stearate salts), glycerides, surfactants, and mixtures thereof.

Surfactants may be, without limitation, cationic, anionic, and nonionic compounds. Exemplary surfactants include polyvinyl alcohol (PVA), gelatin, polyvinyl pyrrolidone (PVP), sodium lauryl sulfate (SLS), and the like.

Glycerides may be, without limitation, monoglyceryl esters, diglyceryl esters, or triglyceryl esters comprising fatty acid having from about 10 to about 22 carbon atoms and glycerol, wherein one or more of the hydroxyl groups of glycerol is substituted by a fatty acid. Exemplary glycerides include glyceryl monostearate, glyceryl distearate, glyceryl tristearate, glyceryl dipalmitate, glyceryl tripalmitate, glyceryl monopalmitate, glyceryl dilaurate, glyceryl trilaurate, glyceryl monolaurate, glyceryl didocosanoate, glyceryl tridocosanoate, glyceryl monodocosanoate, glyceryl monocaproate, glyceryl dicaproate, glyceryl tricaproate, glyceryl monomyristate, glyceryl dimyristate, glyceryl trimyristate, glyceryl monodecenoate, glyceryl didecenoate, glyceryl tridecenoate, and the like, and mixtures thereof. Exemplary water-soluble sugars include, without limitation, mono, and di-saccharides, and polyols, such as sucrose, dextrose, maltodextrin, lactose, mannose, maltose, and mixtures thereof. Exemplary pH-dependent releasing agents include, without limitation, acrylates, acrylate esters, such as methacrylic acid, methylmethacrylates, methylethylacrylates, and mixtures thereof.

The third release control agent can comprise from about 0.2% to about 80% by weight of the final composition, e.g., from about 0.5% to about 10% by weight of the final composition, from about 0.5% to about 15% by weight of the final composition, from about 0.2% to about 20% by weight of the final composition, or from about 5% to about 80% by weight of the final composition. For example, when the third release control agent is an osmotic agent, it can comprise from about 0.5% to about 10% by weight of the final composition. In other embodiments, when the third release control agent is an emulsifier, it can comprise from about 0.2% to about 20% by weight of the final composition. In still other embodiments, when the release control agent is a water-soluble sugar, it can comprise from about 5% to about 80% by weight of the final composition. In other embodiments, if the release control agent is a pH-dependent releasing agent, it can comprise from about 0.5% to about 15% of the final composition.

In another aspect, the invention provides a process for making a modified release tablet composition for oral administration comprising: (a) a dihydropyridine compound or a prodrug or salt thereof; (b) a first release control agent; (c) a second release control agent; and (d) a third release control agent; wherein said second release control agent has a higher viscosity in solution than said first release control agent. The process comprises the steps of (i) providing a composition comprising the dihydropyridine compound or a prodrug or salt thereof, first release control agent, second release control agent, and third release control agent and dry blending the composition; (ii) granulating the dry blend with a polymer to give a granulate; and (iii) tableting the granulate to give a tablet.

In the first step of the process the dihydropyridine compound, first release control agent, second release control agent, and third release control agent are dry mixed thoroughly, in the absence of a solvent, optionally with one or more inert pharmaceutically acceptable excipients to achieve a substantially homogenous mixture. The excipients which may be employed are well known to those skilled in the art and include any conventional pharmaceutically acceptable tableting excipient components including, but not limited to diluents, binders, disintegrants, lubricants, glidants, flow promoting agents, plasticizers, natural and synthetic flavorings and natural and synthetic colorants (e.g., iron oxide and titanium dioxide), or combinations thereof.

Exemplary, non-limiting diluents include starch, lactose, mannitol, kaolin, calcium phosphate or sulfate, inorganic salts, such as sodium chloride, and powdered sugar. Powdered cellulose derivatives may also be used as diluents.

Exemplary, non-limiting binders include substances such as starch, gelatin and sugars, such as lactose, fructose, glucose and the like. Other binders include, for example, acacia, alginates, methylcellulose, polyvinylpyrrolidones (e.g., povidone K29/32, povidone K-17, povidone K-25, povidone K-90) and the like. Finally, polyethylene glycol, ethylcellulose and waxes can also serve as binders.

Exemplary, non-limiting disintegrants include sodium starch glycolate, sodium carboxymethyl cellulose, calcium carboxymethyl cellulose, croscarmellose sodium, crospovidone (polyvinylpolypyrrolidone), methyl cellulose, microcrystalline cellulose, powdered cellulose, starch, pregelatinized starch, and sodium alginate.

Exemplary, non-limiting lubricants include calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc, and zinc stearate.

Exemplary, non-limiting glidants include silicon dioxide, tale, polyethylene glycols (e.g., CARBOWAX SENTRY™ polyethylene glycol 8000; Dow Chemical), and cornstarch. The skilled artisan will recognize that some of these substances can also function as flow promoting agents.

Exemplary, non-limiting plasticizers include polyethylene glycols, triethyl citrate, propylene glycols, diethyl phthalate, dibutyl phthalate, castor oil, triacetin, and others known in the art.

In some embodiments, suitable excipients include, but are not limited to microcrystalline cellulose, dibasic calcium phosphate dihydrate, starch, sodium starch glycolate, crospovidone, croscarmellose sodium, magnesium stearate, lactose, maleic acid, colloidal silicon dioxide, talc, and glyceryl behenate. Additional exemplary excipients include polyethylene glycol, polyvinylpyrrolidone, talc, magnesium stearate, stearic acid, and titanium dioxide. In addition, any of the aforementioned excipients or mixtures thereof may be used in combination with any of the other embodiments described herein.

In the second step of the process, the dry blend comprising the dihydropyridine compound, first release control agent, second release control agent, third release control agent, and the optionally one or more inert pharmaceutically acceptable excipients is granulated in a granulation step in the presence of at least one polymer to give a granulate. A non-limiting example of a polymer that can be used in the granulation step is a polyvinylpyrrolidone such as povidone K29/32, povidone K-17, povidone K-25, povidone K-90, and the like (International Specialty Products). In some embodiments, the granulation step is performed in the presence of a hydroxylic solvent. Exemplary hydroxylic solvents include, without limitation, water, ethanol, isopropanol, t-butanol, and the like. In other embodiments, the granulation step is performed in the presence of at least one polymer, a cellulosic material, polyethylene glycol, or mixtures thereof.

In some embodiments, the granulate is optionally sifted and dried before tableting. For example, the granulate can be sifted using a #30 sieve to provide a sifted granulate. The sifted granulate can subsequently be dried to give a dried granulate such that the solvent used during the granulation step comprises, e.g., less than about 5% by weight, e.g., less than about 2% by weight, less than about 1% by weight, or less than about 0.5% by weight of the granulate. The drying process may provide certain advantages such as content uniformity, ease of handling, etc.

In other embodiments, the dried granulate is optionally mixed with a lubricant prior to tableting.

In the tablet, prior to application of any coating, the dihydropyridine compound and each of the three release control agents are independently distributed substantially uniformly throughout the tablet. In a preferred mode, the tablet composition is effectively homogenous.

After tableting, the tablets thus obtained can optionally be coated with a single coating polymer or a specific mixture of polymers comprising, e.g., a water-impermeable coating polymer, a water-swellable polymer, or a mixture thereof. The coating, however, is substantially free of any dihydropyridine compound. The coating can be applied to the tablet according to methods generally known in the art. For example, a two-step process, or a multi-step process within which the steps may be repeated a sufficient number of times as necessary to build the thickness of the polymeric coating layer to achieve the desired in vitro and in vivo characteristics. The coating substantially completely surrounds the tablet.

Examples of water-impermeable polymers include: ethyl cellulose, propyl cellulose, and the like. Examples of water-swellable polymers include: hydroxypropylmethylcellulose (HMPC), gums, alginates, etc. In some embodiments, the HIPMC is MEITHOCEL™ E15 Premium LV (Dow Chemical), which is a hypromellose that forms a low viscosity solution in water (2% solution in water).

In one aspect, the water-swellable polymer may be a pH-dependent-release polymer such as: anionic polymers of methacrylic acid and methacrylates with a dissolution from pH 5.5 and above (e.g., Eudragit L-100 and Eudragit L 30 D-55); anionic polymers of methacrylic acid and methacrylates with dissolution from pH 6.0 to 7.5 (e.g., Eudragit L100 and Eudragit S100); and copolymers of methacrylic acid, methacrylate and methylmethacrylate with dissolution from pH 7.0 (e.g., Eudragit FS 30 D). These water-swellable polymers are particularly useful in preparation of coatings that facilitate release of active agents, such as dihydropyridine compounds, at certain stages of the intestinal system, with anionic polymers that dissolve at acid-to-neutral pH facilitating release early in the small intestine, and anionic polymers that dissolve at more alkaline pH facilitating release late in the small intestine or in the colon.

In another aspect, the coating mixture comprises HPMC dispersed in an aqueous or substantially nonaqueous solvent. A substantially nonaqueous solvent may be selected form a variety of solvents such as methanol, ethanol, isopropanol, acetone, or a mixture thereof. The HPMC can be selected from one of several grades that are commercially available.

In still another aspect, the coating comprises a cellulosic material, a polyvinylpyrrolidone (Povidone), or mixtures thereof.

The amount of water-insoluble polymer in the coating may range from about 0.5% to about 10% by weight of the tablet, e.g., from about 1% to about 10%, from about 2% to about 8%, from about 2% to about 6%, from about 1% to about 5%, from about 1% to about 3% or from about 2% to about 3% of the weight of the tablet.

The amount of water-swellable polymer in the coating can range from about 0.1% to about 5% by weight of the tablet, e.g., from about 0.5% to about 3%, from about 0.5% to about 2%, or from about 0.5% to about 1.5% by weight of the tablet.

In one aspect, when the coating comprises both a water-insoluble polymer and a water-swellable polymer, the ratio of water-insoluble polymer to the water-swellable polymer can be from about 80/20 to about 20/80, e.g., from about 70/30 to about 30/70, from about 60/40 to about 40/60, or about 50/50.

As discussed previously, after tableting, the formulations of the present invention exhibit a release profile that is approximately zero order. While not being bound by any particular theory, it is believed that the manner in which such a release profile is generated may result from the interplay between the three distinct release control agents. It is believed that the first and second release control agents form a gel matrix that retards the release of the dihydropyridine compound from the matrix, while the third release control agent increases the release rate of the dihydropyridine compound from the matrix, so that overall release from the tableted formulation occurs in an approximately zero order fashion.

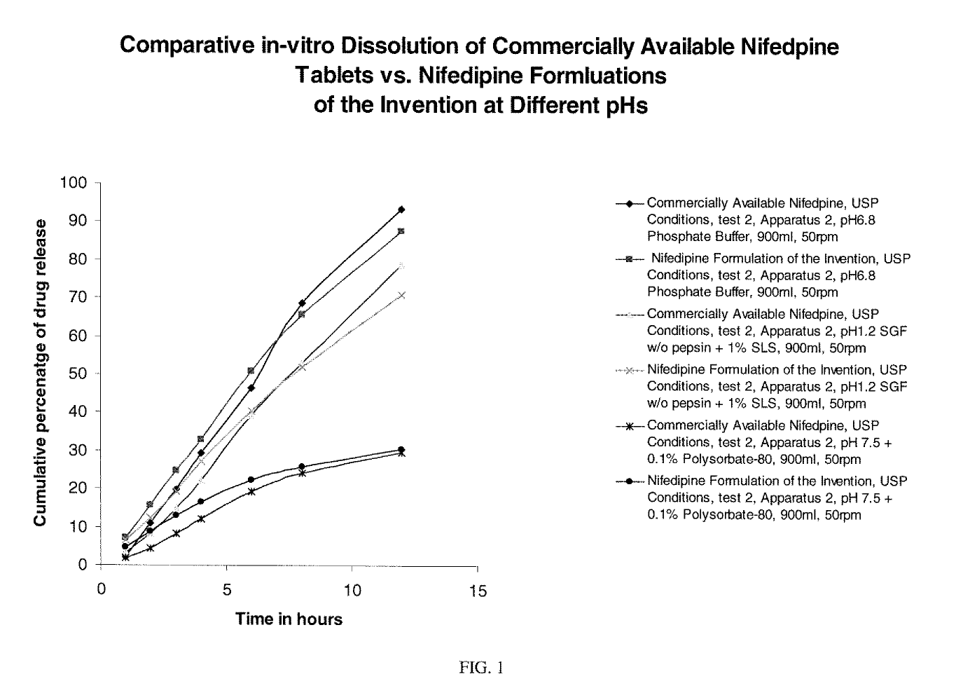

The approximately zero order release of the dihydropyridine compounds of the formulations of the present invention manifests itself as a substantially constant release of the dihydropyridine compound as a function of time, as shown in FIG. 1. In FIG. 1, the approximately zero order release observed for nifedipine, for example, is substantially the same as for commercially available formulations of nifedipine that are formulated with a conventional mixture of excipients and rely on an additional coating to achieve the observed zero order release profile. The inventive formulation, in contrast, does not rely on a coating to effect the same release profile, although tablets comprising the inventive formulation can optionally comprise a controlled release coating.

In one aspect, therefore, the invention provides a tablet composition for oral administration comprising:

(a) a dihydropyridine compound or a prodrug or salt thereof,
(b) a first release control agent;
(c) a second release control agent; and
(d) an osmotic agent
wherein the dihydropyridine compound or a prodrug or salt thereof is provided in a micronized, crystalline or amorphous form when the tablet is formed; and
wherein, in vitro at a pH of less than 7 and in the presence of 1% sodium lauryl sulfate, the composition releases from about 25% to about 50%%, or, e.g., from about 25% to about 55% of the dihydropyridine compound or a prodrug or salt thereof after 6 hours and not less than about 65% or not less than 75% of the dihydropyridine compound or a prodrug or salt thereof after 12 hours.

In another aspect, the invention provides a tablet composition for oral administration comprising:

(a) a dihydropyridine compound or a prodrug or salt thereof;
(b) a first release control agent;
(c) a second release control agent; and
(d) an osmotic agent
wherein the dihydropyridine compound or a prodru, or salt thereof is provided in a micronized, crystalline or amorphous form when the tablet is formed; and
wherein, in vitro at a pH of greater than 7, the composition releases from about 10% to about 20% of the dihydropyridine compound or a prodrug or salt thereof after 6 hours and from about 25% to about 35% of the dihydropyridine compound or a prodrug or salt thereof after 12 hours.

In another aspect, the invention provides a tablet composition for oral administration comprising:

(a) a dihydropyridine compound or a prodrug or salt thereof (b) a first release control agent;
(c) a second release control agent; and
(d) an osmotic agent
wherein the dihydropyridine compound or a prodrug or salt thereof is provided in a micronized, crystalline or amorphous form when the tablet is formed; and
wherein, in vitro at a pH of 1.2 (e.g., simulated gastric fluid (SGF)), in the presence of 1% sodium lauryl sulfate, using a U.S. Pharmacopeia (USP) Type II apparatus operating at 50 rpm, the composition releases from about 1% to about 10% of the dihydropyridine compound or a prodrug or salt thereof after one hour; from about 10% to about 30% after four hours; from about 40% to about 65% after eight hours; and from about 70% to about 90% after 12 hours.

In still another aspect, the invention provides a tablet composition for oral administration comprising.

(a) a dihydropyridine compound or a prodrug or salt thereof;
(b) a first release control agent;
(c) a second release control agent; and
(d) an osmotic agent
wherein the dihydropyridine compound or a prodrug or salt thereof is provided in a micronized, crystalline or amorphous form when the tablet is formed; and
wherein, in vitro at a pH of 4.5 (e.g., acetate buffer), in the presence of 1% sodium lauryl sulfate, using a USP Type II apparatus operating at 50 rpm, the composition releases from about 1% to about 15% of the dihydropyridine compound or a prodrug or salt thereof after one hour; from about 7% to about 20% after two hours; from about 20% to about 40% after four hours; from about 40% to about 75% after eight hours; and from about 70% to about 90% after 12 hours.

In yet another aspect, the invention provides a tablet composition for oral administration comprising:

(a) a dihydropyridine compound or a prodrug or salt thereof;
(b) a first release control agent;
(c) a second release control agent; and
(d) an osmotic agent
wherein the dihydropyridine compound or a prodrug or salt thereof is provided in a micronized, crystalline form when the tablet is formed;
wherein, in vitro at a pH of 6.8 (e.g., phosphate buffer), in the presence of 1% sodium lauryl sulfate, using a USP Type II apparatus operating at 50 rpm, the composition releases from about 1% to about 10% of the dihydropyridine compound or a prodrug or salt thereof after one hour; from about 7% to about 20% after two hours; from about 20% to about 40% after four hours; from about 50% to about 80% after eight hours; and from about 75% to about 90% after 12 hours.

### EXAMPLES

The following examples are provided to illustrate the present invention, and should not be construed as a limitation thereof. All percentages are in percent by weight of the tablet unless otherwise indicated. Unless indicated otherwise, disintegration tests are carried out according to the standard USP procedures for testing the disintegration of tablets.

### Example 1

### Nifedipine Tablets

A solution of 5% Povidone K-29/32 (210 g; International Specialty Products) was prepared in IPA (3,201 g) in a suitable container. The solution was stirred at approximately 30° C. for about 2 hours. Nifedipine (1,500 g; micronized, >4 m²/g surface area; Zambon Co., S.P.A.) was dry blended in the absence of a solvent with microcrystalline cellulose (400 g; EMCOCEL™ 90M; JRS Pharma Inc.), lactose (760 g; Kerry BioscienceCallahan), sodium chloride (50 g; #100; Mortan Salts), HPMC K100LV (1,580 g; Methocel K100LV PremiumDow Chemical), and HPMC K4M (450 g; Methocel K4M Premium CRDow Chemical) in a high shear granulator (propeller set at 85 rpm; chopper set at 75 rpm; time 30 minutes).

The nifedipine mixture was then granulated by adding the povidone solution to the nifedipine mixture at a rate of 15 grams/minute. The granulated nifedipine mixture was sifted through a #30 sieve and was subsequently dried in a tray drier for approximately 12 hours at 45° C. until the solvent content was less than 0.5%. The granulated material was subsequently lubricated with magnesium stearate (50 g; FACI) in a double cone blender. The dried nifedipine mixture was then tableted to a size of from about 5 mm in diameter to about 10 mm in diameter (e.g., from about 6 mm in diameter to about 10 mm in diameter, and from about 7 mm in diameter to about 10 mm in diameter) with a force sufficient to make tablets of a hardness of from about 4 kp to about 10 kp (e.g., from about 6 kp to about 10 kp, from about 4 kp to about 8 kp, and from about 4 kp to about 6 kp) using tableting techniques well known in the art.

Tablets made using the foregoing procedure can optionally be coated with a coating polymer. An exemplary coating polymer can be prepared as an aqueous solution/suspension of the polymer, namely, METHOCEL™ E15 Premium LV (104 g; Dow Chemical). To this solution/suspension can be added a glidant, namely, CARBOWAX SENTRY™ polyethylene glycol 8000 (20 g; Dow Chemical). The solution/suspension can be made using 2,086 g of water (USP).

Tablets made using the foregoing procedure can optionally be colored. For example, the color can be formulated by homogenizing iron oxide (104 g; Colorcon) and titanium dioxide (20 g; American International Chemical, Inc.) in 834 g of water.

### Example 2

### In Vitro Dissolution Profiles for Uncoated Nifedipine Tablets Made According to the Present Invention Compared to Commercially Available Nifedipine Tablets

The in vitro dissolution for nifedipine tablets made according to Example 1 (9 mm tablet) was compared to commercially available nifedipine tablets at varying pHs, and the resultant profiles are shown in FIG. 1. The tablets that were used in all cases were 90 mg tablets. The dissolution profiles were determined using a U.S. Pharmacopeia (USP) Type II apparatus operating at 50 rpm at 37° C.±0.5 under the following conditions: (i) pH 6.8; 900 mL of phosphate buffer with 1% sodium lauryl sulfate (SLS); (ii) pH 7.5; 900 mL water; 0.1% polysorbate-80; 900 mL of water; and (iii) pH 1.2 using simulated gastric fluid (SGF) without pepsin; 1% SLS; 900 mL of water. It is evident from the dissolution profiles shown in FIG. 1 that at each pH, the dissolution profile for nifedipine tablets made according to the present invention is approximately zero order and very similar to the dissolution profile of commercially available nifedipine tablets at the same pH.

### Example 3

### In Vitro Dissolution Profiles for Coated Nifedipine Tablets Made According to the Present Invention Compared to Commercially Available Nifedipine Tablets

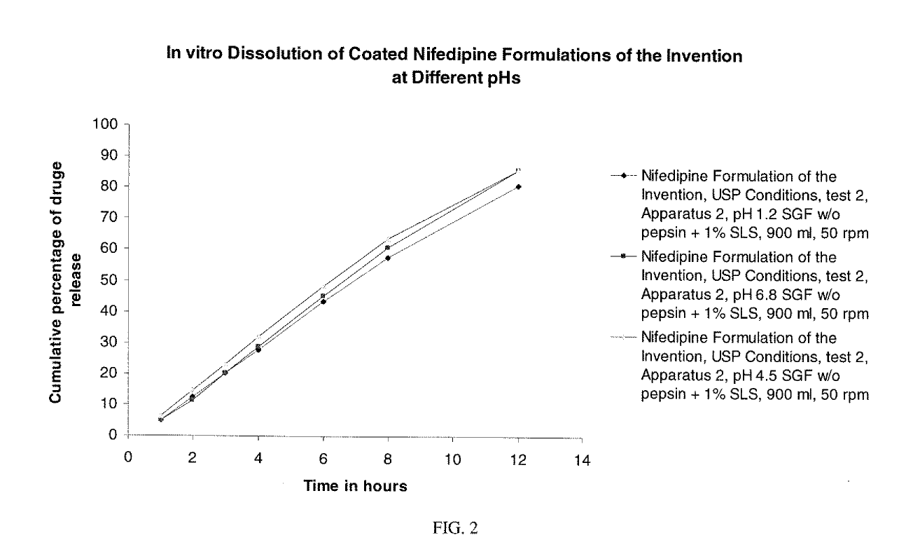

The in vitro dissolution for nifedipine coated tablets made according to Example 1 (9 mm tablet) was obtained at varying pHs, and the resultant profiles are shown in FIG. 2. The tablets that were used in all cases were 305.92 mg tablets and were coated using an aqueous solution/suspension of METHOCEL™ E15 Premium LV (104 g; Dow Chemical). The coating also contained CARBOWAX SENTRY™ polyethylene glycol 8000 (20 g; Dow Chemical). The dissolution profiles were determined using a U.S. Pharmacopeia (USP) Type II apparatus operating at 50 rpm at 37° C.±0.5 under the following conditions: (i) pH 6.8; 900 mL of simulated intestinal fluid (SW) without pepsin and 1% SLS; 900 mL of water; (ii) pH 4.5; 900 mL of an acetate buffer and 1% SLS; and (iii) pH 1.2 using simulated gastric fluid (SGF) without pepsin; 1% SLS; 900 mL of water. It is evident from the dissolution profiles shown in FIG. 2 that at each pH, the dissolution profile for nifedipine tablets made according to the present invention is approximately zero order.

### Example 4

### In Vitro Dissolution Profiles for Uncoated Nifedipine Tablets Made According to the Present Invention Compared to Commercially Available Nifedipine Tablets

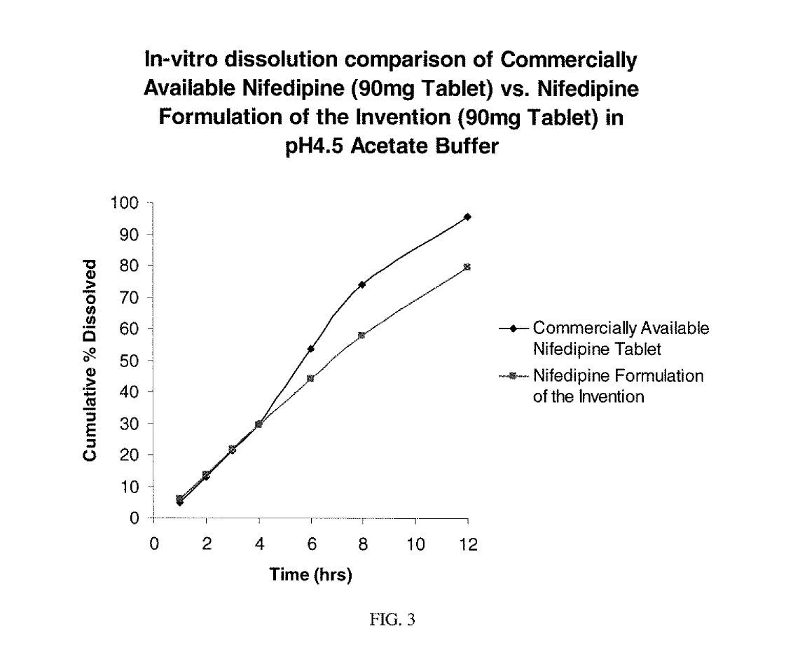

The in vitro dissolution for nifedipine tablets made according to Example 1 (9.0 mm tablets) was compared to commercially available nifedipine tablets at pH 4.5 (acetate buffer), and the resultant profiles are shown in FIG. 3. The tablets that were used in all cases were 90 mg tablets. The dissolution profiles were determined using a U.S. Pharmacopeia (USP) Type II apparatus operating at 50 rpm at 37° C.±0.5. It is evident from the dissolution profiles shown in FIG. 2 that at pH 4.5, the dissolution profile for nifedipine tablets made according to the present invention is approximately zero order and very similar to the dissolution profile of commercially available nifedipine tablets.

### Example 5

### Analysis Procedure for In Vitro Dissolution Tests

Standard solutions: an accurately weighed quantity of USP Nifedipine RS was dissolved in methanol to obtain a stock solution having a known concentration of about 1.11 mg per mL. The stock solution was diluted quantitatively and stepwise with a mixture of acetonitrile and water (70:30) to obtain a number of solutions having a known concentration of nifedipine.

Chromatographic system: the liquid chromatograph was equipped with a 350-nm detector and a 4.0-mm×125-mm column that contained 3-μm packing. The mobile phase was a filtered and degassed mixture of acetonitrile and water (70:30). The flow rate of the mobile phase was about 1.5 mL per minute. The column was maintained at about 40° C. Each standard solution of varying concentration of nifedipine was chromatographed and the peak responses were recorded to generate a concentration correlation curve. The column efficiency was not less than 2000 theoretical plates; the tailing factor was not more than 1.5; and the relative standard deviation for replicate injections was not more than 2.0%. The injection volumes were about 20 μL.

### Example 6

### Pharmacokinetic Parameters for Healthy Human Volunteers Using Extended Release (ER), 90 mg Nifedipine Tablets Under Fasted and Fed Conditions

The tablets used to generate the pharmacokinetic parameters in Tables 2 and 3 are the same tablets described in Example 1 comprising a METHOCEL™ E15 Premium LV and CARBOWAX SENTRY™ polyethylene glycol 8000 coating. The area under the curve at the last time point collected (AUCₗₐₛₜ) and at infinity (AUC_{∞}), as well as the maximum plasma concentration (Cₘₐₓ), averaged from data obtained for twelve subjects is shown in Tables 2 and 3 under fed and fasted conditions, respectively.

TABLE 2
nifedipine ER 90 mg tablets under fed conditions

| | | | Intra CV % |
|---|---|---|---|
| Ln(AUC_{∞}) | 2608.15 | hr * ng/ml | 12.67% |
| Ln(AUCₗₐₛₜ) | 2504.06 | hr * ng/ml | 16.32% |
| Ln(Cₘₐₓ) | 398.61 | ng/ml | 39.50% |

TABLE 3
nifedipine ER 90 mg tablets under fasting conditions

| | | | Intra CV % |
|---|---|---|---|
| Ln(AUC_{∞}) | 3283.52 | hr * ng/ml | 14.87% |
| Ln(AUCₗₐₛₜ) | 3122.58 | hr * ng/ml | 15.53% |
| Ln(Cₘₐₓ) | 305.38 | ng/ml | 38.50% |

The average pharmacokinetic parameters shown in Tables 1 and 2 were obtained by determining the pharmacokinetic parameters for each individual study subject and subsequently averaging the values obtained.

The pharmacokinetic parameters shown in Table 3 (fasting) demonstrate that the coated tablets made according to Example 1 appear to be bioequivalent to commercially available nifedipine tablets (e.g., ADALAT® CC). As used herein, the term “bioequivalent” means that the values for AUC_{∞} and Cₘₐₓ of the tablets made according to the invention are between 80% and 125% of the AUC_{∞} and Cₘₐₓ values of commercially available nifedipine tablets. The AUC_{∞} for ADALAT® CC (fasting) was determined to be 3505.96 hr*ng/ml and the Cₘₐₓ (fasting) was determined to be 284.96 ng/1 ml.

### Example 7

### Nifedipine 90 mg Tablet Strength

Weighed 20.g of Nifedipine Micronized (>4 sqm/g), 14.9 g of Lactose and 11.0 g of Micro crystalline cellulose powder, 24.5 g Hydroxy Propyl methyl cellulose substitution type 2208 with a nominal viscosity of 100 cP, 24.4 g Hydroxy Propyl methyl cellulose substitution type 2208 with a nominal viscosity of 4000 cP were mixed properly in a Rapid Mixing Granulator. Dissolved 4.2 g of Povidone K-30 into a Isopropyl alcohol under continuous stirring, until clear solution is obtained. The preblend of Nifedipine excipient dry mixture was granulated with the solution of Povidone K-30 in a Rapid mixing granulator. The wet mass obtained after granulation was dried using fluid bed processor until moisture content is NMT 1.0%. The dried granules were passed through ASTM #30 screen. The granules were lubricated with 0.19 g of magnesium stearate in a mixer for 5 min. The blend was compressed on a tabletting machine with a punch size of 11.0 mm and weight of 450 mg. Hardness 5.0 KN, friability less than 1.0%.

### Example 8

### Nifedipine 90 mg Tablet Strength

Weighed 20 g of nifedipine micronized (>4 sqn/g), 14.9 g of lactose and 11.0 g of micro crystalline cellulose powder, 34.2 g hydroxy propyl methyl cellulose substitution type 2208 with a nominal viscosity of 100 cP, 15.7 g Hydroxy Propyl methyl cellulose substitution type 2208 with a nominal viscosity of 4000 cP were mixed properly in a rapid mixing granulator. Dissolved 4.2 g of povidone K-30 into a isopropyl alcohol under continuous stirring, until clear solution is obtained. The preblend of nifedipine-excipient dry mixture was granulated with the solution of povidone K-29/32 in a rapid mixing granulator. The wet mass obtained after granulation was dried using fluid bed processor until moisture content is NMT 1.0%. The dried granules were passed through ASTM #30 screen. The granules were lubricated with 0.19 g of magnesium stearate in a mixer for 5 min. The blend was compressed on a tabletting machine with a punch size of 110.0 mm and weight of 450 mg. Hardness 5.0 KN, friability less than 1.0%.

### Example 9

### Nifedipine 60 mg Tablet Strength

Weighed 20 g of nifedipine micronized (>4 sqm/g), 20.0 g of lactose and 11.0 g of micro crystalline cellulose powder, 1.0 g Sodium chloride (passed through #60 screen), 31.6 g hydroxy propyl methyl cellulose substitution type 2208 with a nominal viscosity of 100 cP, 11.2 g hydroxy propyl methyl cellulose substitution type 2208 with a nominal viscosity of 4000 cP were mixed properly in a rapid mixing granulator. Dissolved 4.2 g of povidone K-30 into a isopropyl alcohol under continuous stirring, until clear solution is obtained. The preblend of nifedipine-excipient dry mixture was granulated with the solution of povidone K-30 in a rapid mixing granulator. The wet mass obtained after granulation was dried using fluid bed processor until moisture content is NMT 1.0%. The dried granules were passed through ASTM #30 screen. The granules were lubricated with 0.19 g of magnesium stearate in a mixer for 5 min. The blend was compressed on a tabletting machine with a punch size of 9.0 mm, standard concave shape tablets, and weight of 300 mg. Hardness 5.0 KN, Friability less than 1.0%.

### Example 10

### Nifedipine 90 mg Tablet Strength with Film Coating

Weighed 30 g of nifedipine micronized (>4 sqm/g), 15.2 g of lactose and 8.0 g of micro crystalline cellulose powder, 1.0 g sodium chloride, 31.6 g hydroxy propyl methyl cellulose substitution type 2208 with a nominal viscosity of 100 cP, 9.0 g hydroxy propyl methyl cellulose substitution type 2208 with a nominal viscosity of 4000 cP were mixed properly in a rapid mixing granulator. Dissolved 4.2 g of povidone K-30 into a isopropyl alcohol under continuous stirring, until clear solution is obtained. The preblend of nifedipine-excipient dry mixture was granulated with the solution of povidone K-30 in a rapid mixing granulator. The wet mass obtained after granulation was dried using fluid bed processor until moisture content is NMT 1.0%. The dried granules were passed through ASTM #30 screen. The granules were lubricated with 0.19 g of magnesium stearate in a mixer for 5 min. The blend was compressed on a tabletting machine with a punch size of 9.0 mm, standard concave shape tablets, and weight of 300 mg. Hardness 5.0 KN, friability less than 1.0%. 0.81 g of hydroxy propyl methyl cellulose E15LV, was dissolved in purified water under continuous stirring. 0.16 g of polyethylene glycol 8000 was added into the above solution. 0.16 g of titaniumdioxide and 0.81 g iron oxide color was homogenized separately and added to the above coating solution under continuous stirring. The solution was coated onto tablets in a perforated or conventional coating pan.

### Example 11

### Nifedipine 30 mg Tablet Strength with Film Coating

Weighed 10.0 g of nifedipine micronized (>4 sqm/g), 20.2 g of lactose and 13.0 g of micro crystalline cellulose powder, 1.0 g Sodium chloride, 36.6 g hydroxy propyl methyl cellulose substitution type 2208 with a nominal viscosity of 100 cP, 14.0 g hydroxy propyl methyl cellulose substitution type 2208 with a nominal viscosity of 4000 cP were mixed properly in a rapid mixing granulator. Dissolved 4.2 g of povidone K-30 into a isopropyl alcohol under continuous stirring, until clear solution is obtained. The preblend of nifedipine-excipient dry mixture was granulated with the solution of povidone K-30 in a rapid mixing granulator. The wet mass obtained after granulation was dried using fluid bed processor until moisture content is NMT 1.0%. The dried granules were passed through ASTM #30 screen. The granules were lubricated with 0.19 g of magnesium stearate in a mixer for 5 min. The blend was compressed on a tabletting machine with a punch size of 9.0 mm, standard concave shape tablets, and weight of 300 mg. Hardness 5.0 KN, friability less than 1.0%. 0.81 g of hydroxy propyl methyl cellulose E15LV, was dissolved in purified water under continuous stirring. 0.16 g of polyethylene glycol 8000 was added into the above solution. 0.16 g of titaniumdioxide and 0.81 g iron oxide color was homogenized separately and added to the above coating solution under continuous stirring. The solution was coated onto tablets in a perforated or conventional coating pan.

While the foregoing description includes details and specific examples, it is to be understood that these have been included for purposes of explanation only, and are not to be interpreted as limitations of the present invention. Modifications to the embodiments described herein can be made without departing from the spirit and scope of the invention, which is intended to be encompassed by the following claims and their legal equivalents.

What is claimed is:

1. An extended release tablet composition for oral administration comprising:
(a) a dihydropyridine compound or a prodrug or salt thereof;
(b) a first release control agent;
(c) a second release control agent; and
(d) a third release control agent;
wherein said second release control agent has a higher viscosity in solution than said first release control agent.

2. The composition of claim 1, wherein the dihydropyridine compound or a prodrug or salt thereof is provided in a micronized, crystalline or amorphous form when the tablet is formed.

3. The composition of claim 1, wherein the dihydropyridine compound is amlodipine, aranidipine, azelnidipine, barnidipine, benidipine, cilnidipine, clevidipine, cronidipine, darodipine, dexniguldipine, efonidipine, elnadipine, elgodipine, felodipine, flordipine, furnidipine, iganidipine, isradipine, lacidipine, lemildipine, lercanidipine, manidipine, mesuldipine, nicardipine, nifedipine, niguldipine, nimodipine, niludipine, nilvadipine, nimodipine, nisoldipine, nitrendipine, olradipine, oxodipine, palonidipine, pranidipine, sagandipine, sornidipine, teludipine, tiamdipine, trombodipine, watanidipine, or a prodrug or salt thereof or mixtures thereof.

4. The composition of claim 1, wherein the first release control agent comprises a cellulosic material.

5. The composition of claim 4, wherein the cellulosic material comprises methylcellulose, hydroxypropyl cellulose (HPC), hydroxyethyl cellulose (HPC), hypromellose, or mixtures thereof.

6. The composition of claim 1, wherein the first release control agent comprises a hypromellose that forms a low viscosity solution in water.

7. The composition of claim 1, wherein the second release control agent comprises a cellulosic material.

8. The composition of claim 7, wherein the cellulosic material comprises hydroxypropyl cellulose (HPC), hydroxyethyl cellulose (HEC), hypromellose, or mixtures thereof.

9. The composition of claim 1, wherein the second release control agent comprises a hypromellose that forms a high viscosity solution in water.

10. The composition of claim 1, wherein the third release control agent increases the rate of release of the dihydropyridine compound or prodrug or salt thereof.

11. The composition of claim 1, wherein said third release control agent is an osmotic agent, an emulsifier, a water-soluble sugar, a pH-dependent releasing agent, or a mixture thereof.

12. The composition of claim 11, wherein said osmotic agent is sodium chloride, potassium monophosphate, or fumaric acid.

13. The composition of claim 11, wherein said osmotic agent is sodium chloride.

14. The composition of claim 11, wherein said emulsifier has a high hydrophilic lipophilic balance (HLB).

15. The composition of claim 14, wherein said emulsifier has an HLB of greater than 10.

16. The composition of claim 11, wherein said emulsifier is a polyoxylated sorbitan-based compound, a fatty acid salt, a glyceride, a surfactant, or mixtures thereof.

17. The composition of claim 11, wherein said emulsifier is polysorbate 80.

18. The composition of claim 11, wherein said emulsifier is sodium lauryl sulfate.

19. The composition of claim 11, wherein said emulsifier is a glyceride.

20. The composition of claim 11, wherein said water-soluble sugar is a monosaccharide, a di-saccharide, a polyol, or mixtures thereof.

21. The composition of claim 11, wherein said water-soluble sugar is sucrose, dextrose, maltodextrin, lactose, mannose, maltose, or mixtures thereof.

22. The composition of claim 11, wherein said pH-dependent releasing agent is an acrylate, an acrylate ester, a methylmethacrylate, a methylethylacrylate, or mixtures thereof.

23. The composition of claim 1, wherein the composition further comprises one or more excipient components selected from the group consisting of diluents, binders, lubricants, glidants or combinations thereof.

24. The composition of claim 23, wherein the diluents comprise microcrystalline cellulose, lactose, or mixtures thereof.

25. The composition of claim 23, wherein the binder comprises a polyvinyl pyrrolidone.

26. The composition of claim 25, wherein the binder comprises povidone 29/32, povidone K-17, povidone K-25, povidone K-90, or mixtures thereof.

27. The composition of claim 26, wherein the binder comprises povidone K29/32.

28. The composition of claim 23, wherein the lubricant comprises calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate, or mixtures thereof.

29. The composition of claim 28, wherein the lubricant comprises magnesium stearate.

30. The composition of claim 23, wherein the glidant comprises silicon dioxide, talc, polyethylene glycols, cornstarch, or mixtures thereof.

31. The composition of claim 30, wherein the glidant comprises polyethylene glycols.

32. The composition of claim 1, wherein the dihydropyridine compound or a prodrug or salt thereof is substantially homogeneously dispersed throughout said tablet.

33. The composition of one of claims 1 or 32, wherein said tablet is optionally coated with a coating and wherein said coating is substantially free of the dihydropyridine compound or a prodrug or salt thereof.

34. The composition of claim 33, wherein said coating comprises a cellulosic material, povidone, or mixtures thereof.

35. The composition of claim 34, wherein the cellulosic material comprises hydroxypropyl cellulose (HPC), hydroxyethyl cellulose (HEC), hypromellose, or mixtures thereof.

36. A process for making the tablet composition of claim 1, comprising:
(a) providing a composition comprising the dihydropyridine compound or a prodrug or salt thereof, first release control agent, second release control agent, and third release control agent and dry blending said composition;
(b) granulating said dry blend with a polymer, a cellulosic material, polyethylene glycol, or mixtures thereof, to give a granulate; and
(c) tableting the granulate composition to give a tablet.

37. The process of claim 36, wherein the dihydropyridine compound or a prodrug or salt thereof is provided in a micronized, crystalline or amorphous form when the tablet is formed.

38. The process of claim 36, wherein the cellulosic material comprises hydroxypropyl cellulose (HPC), hydroxyethyl cellulose (HEC), hypromellose, or mixtures thereof.

39. The process of claim 36, wherein the dihydropyridine compound is amlodipine, aranidipine, azelnidipine, barnidipine, benidipine, cilnidipine, clevidipine, cronidipine, darodipine, dexniguldipine, efonidipine, elnadipine, elgodipine, felodipine, flordipine, furnidipine, iganidipine, isradipine, lacidipine, lemildipine, lercanidipine, manidipine, mesuldipine, nicardipine, nifedipine, niguldipine, nimodipine, niludipine, nilvadipine, nimodipine, nisoldipine, nitrendipine, olradipine, oxodipine, palonidipine, pranidipine, sagandipine, sornidipine, teludipine, tiamdipine, trombodipine, watanidipine, or mixtures thereof or a prodrug or salt thereof.

40. The process of claim 36, wherein the polymer is a polyvinylpyrrolidone polymer.

41. The process of claim 36, wherein said tablet is optionally coated with a coating that is substantially free of the dihydropyridine compound or a prodrug or salt thereof.

42. The process of claim 36, wherein the first release control agent comprises a cellulosic material.

43. The process of claim 42, wherein the cellulosic material comprises methyl cellulose, hydroxypropyl cellulose (HPC), hydroxyethyl cellulose (HEC), hypromellose, or mixtures thereof.

44. The process of claim 36, wherein the first release control agent comprises a hypromellose that forms a low viscosity solution in water.

45. The process of claim 36, wherein the second release control agent comprises a cellulosic material.

46. The process of claim 45, wherein the cellulosic material comprises hydroxypropyl cellulose (HPC), hydroxyethyl cellulose (HEC), hypromellose, or mixtures thereof.

47. The process of claim 36, wherein the second release control agent comprises a hypromellose that forms a high viscosity solution in water.

48. The process of claim 35, wherein the third release control agent increases the rate of release of the dihydropyridine compound.

49. The process of claim 36, wherein said third release control agent is an osmotic agent, an emulsifier, a water-soluble sugar, a pH-dependent releasing agent, or a mixture thereof.

50. The process of claim 49, wherein said osmotic agent is sodium chloride, potassium monophosphate, or fumaric acid.

51. The process of claim 49, wherein said osmotic agent is sodium chloride.

52. The process of claim 49, wherein said emulsifier has a high hydrophilic lipophilic balance (HLB).

53. The process of claim 52, wherein said emulsifier has an HLB of greater than 10.

54. The process of claim 49, wherein said emulsifier is a polyoxylated sorbitan-based compound, a fatty acid salt, a glyceride, a surfactant, or mixtures thereof.

55. The process of claim 54, wherein said emulsifier comprises a polyoxylated sorbitan-based compound.

56. The process of claim 55, wherein said emulsifier comprises polysorbate 80.

57. The process of claim 49, wherein said emulsifier is sodium lauryl sulfate.

58. The process of claim 49, wherein said emulsifier is a glyceride.

59. The process of claim 49, wherein said water-soluble sugar is a monosaccharide, a di-saccharide, a polyol, or mixtures thereof.

60. The process of claim 49, wherein said water-soluble sugar is sucrose, dextrose, maltodextrin, lactose, mannose, maltose, or mixtures thereof.

61. The process of claim 49, wherein said pH-dependent releasing agent is an acrylate, an acrylate ester, a methylmethacrylate, a methylethylacrylate, or mixtures thereof.

62. The process of claim 36, wherein the composition further comprises one or more excipient components selected from the group consisting of diluents, binders, lubricants, glidants or combinations thereof.

63. A tablet composition for oral administration comprising:
(a) a dihydropyridine compound or a prodrug or salt thereof;
(b) a first release control agent;
(c) a second release control agent; and
(d) an osmotic agent;
wherein the dihydropyridine compound or a prodrug or salt thereof is provided in a micronized, crystalline or amorphous form when the tablet is formed; and
wherein, in vitro at a pH of less than 7 and in the presence of 1% sodium lauryl sulfate, the composition releases from about 25% to about 50% of the dihydropyridine compound or a prodrug or salt thereof after 6 hours and not less than about 75% of the dihydropyridine compound or a prodrug or salt thereof after 12 hours.

64. A tablet composition for oral administration comprising:
(a) a dihydropyridine compound or a prodrug or salt thereof;
(b) a first release control agent;
(c) a second release control agent; and
(d) an osmotic agent;
wherein the dihydropyridine compound or a prodrug or salt thereof is provided in a micronized, crystalline or amorphous form when the tablet is formed; and
wherein, in vitro at a pH of greater than 7, the composition releases from about 10% to about 20% of the dihydropyridine compound or a prodrug or salt thereof after 6 hours and from about 25% to about 35% of the dihydropyridine compound or a prodrug or salt thereof after 12 hours.

65. A tablet composition for oral administration comprising:
(a) a dihydropyridine compound or a prodrug or salt thereof;
(b) a first release control agent;
(c) a second release control agent; and
(d) an osmotic agent;
wherein the dihydropyridine compound or a prodrug or salt thereof is provided in a micronized, crystalline or amorphous form when the tablet is formed; and
wherein, in vitro at a pH of 1.2, in the presence of 1% sodium lauryl sulfate, using a USP Type II apparatus operating at 50 rpm and comprising 900 mL of water, the composition releases from about 1% to about 10% of the dihydropyridine compound or a prodrug or salt thereof after one hour; from about 10% to about 30% after four hours; from about 40% to about 65% after eight hours; and from about 70% to about 90% after 12 hours.

66. A tablet composition for oral administration comprising;
(a) a dihydropyridine compound or a prodrug or salt thereof;
(b) a first release control agent;
(c) a second release control agent; and
(d) an osmotic agent;
wherein the dihydropyridine compound or a prodrug or salt thereof is provided in a micronized, crystalline or amorphous form when the tablet is formed; and
wherein, in vitro at a pH of 4.5, in the presence of 1% sodium lauryl sulfate, using a USP Type II apparatus operating at 50 rpm and comprising 900 mL of water, the composition releases from about 1% to about 15% of the dihydropyridine compound or a prodrug or salt thereof after one hour; from about 7% to about 20% after two hours; from about 20% to about 40% after four hours; from about 40% to about 75% after eight hours; and from about 70% to about 90% after 12 hours.

67. A tablet composition for oral administration comprising:
(a) a dihydropyridine compound or a prodrug or salt thereof;
(b) a first release control agent;
(c) a second release control agent; and
(d) an osmotic agent;
wherein the dihydropyridine compound or a prodrug or salt thereof is provided in a micronized, crystalline or amorphous form when the tablet is formed; and
wherein, in vitro at a pH of 6.8, in the presence of 1% sodium lauryl sulfate, using a USP Type II apparatus operating at 50 rpm and comprising 900 mL of aqueous phosphate buffer, the composition releases from about 1% to about 10% of the dihydropyridine compound or a prodrug or salt thereof after one hour; from about 7% to about 20% after two hours; from about 20% to about 40% after four hours; from about 50% to about 80% after eight hours; and from about 75% to about 90% after 12 hours.

68. The composition of any one of claims 63, 64, 65, 66, or 67, wherein the dihydropyridine compound is amlodipine, aranidipine, azelnidipine, barnidipine, benidipine, cilnidipine, clevidipine, cronidipine, darodipine, dexniguldipine, efonidipine, elnadipine, elgodipine, felodipine, flordipine, furnidipine, iganidipine, isradipine, lacidipine, lemildipine, lercanidipine, manidipine, mesuldipine, nicardipine, nifedipine, niguldipine, nimodipine, niludipine, nilvadipine, nimodipine, nisoldipine, nitrendipine, olradipine, oxodipine, palonidipine, pranidipine, sagandipine, sornidipine, teludipine, tiamdipine, trombodipine, watanidipine, or a prodrug or salt thereof or mixtures thereof.

69. The composition of claim 68, wherein the dihydropyridine compound or a prodrug or salt thereof is provided in a micronized, crystalline or amorphous form when the tablet is formed.

70. The composition of claim 68, wherein the first release control agent comprises a cellulosic material.

71. The composition of claim 70, wherein the cellulosic material comprises methylcellulose, hydroxypropyl cellulose (HPC), hydroxyethyl cellulose (HEC), hypromellose, or mixtures thereof.

72. The composition of claim 68, wherein the first release control agent comprises a hypromellose that forms a low viscosity solution in water.

73. The composition of claim 68, wherein the second release control agent comprises a cellulosic material.

74. The composition of claim 73, wherein the cellulosic material comprises hydroxypropyl cellulose (HPC), hydroxyethyl cellulose (HEC), hypromellose, or mixtures thereof.

75. The composition of claim 68, wherein the second release control agent comprises a hypromellose that forms a high viscosity solution in water.

76. The composition of claim 68, wherein the osmotic agent increases the rate of release of the dihydropyridine compound or prodrug or salt thereof.

77. The composition of claim 68 wherein said osmotic agent is sodium chloride, potassium monophosphate, or fumaric acid.

78. The composition of claim 68, wherein said osmotic agent is sodium chloride.

79. The composition of claim 68, wherein the composition further comprises one or more excipient components selected from the group consisting of diluents, binders, lubricants, glidants or combinations thereof.

80. The composition of claim 68, wherein said tablet is optionally coated with a coating that is substantially free of the dihydropyridine compound or a prodrug or salt thereof.
